(19) United States
(12) Patent Application Publication
(10) Pub. No.: US 2017/0367806 A1
(43) Pub. Date: Dec. 28, 2017

(54) TISSUE REPAIR IMPLANTS AND METHODS FOR MAKING AND USING SAME

(71) Applicant: VISCUS BIOLOGICS, LLC, Cleveland, OH (US)

(72) Inventors: Peter H. Gingras, Cleveland, OH (US); Robert M. Priest, Cleveland, OH (US)

(73) Assignee: VISCUS BIOLOGICS, LLC, Cleveland, OH (US)

(21) Appl. No.: 15/533,446

(22) PCT Filed: Dec. 7, 2015
(86) PCT No.: PCT/US15/64227

§ 371 Date: Jun. 6, 2017

Related U.S. Application Data
(60) Provisional application No. 62/088,754, filed on Dec. 8, 2014.

(51) Int. Cl. A61F 2/00; A61L 31/10

(52) U.S. Cl. CPC A61F 2/0063; A61L 31/10; A61F 2210/0076; A61F 2002/0068; A61F 2250/0051

(57) ABSTRACT

One aspect of the present disclosure relates to a tissue repair implant comprising a first layer of extracellular matrix and a second support layer of biocompatible material securely attached to the first layer at one or more fixation points. At least one of the fixation points comprises a first projection that is associated with the first layer and securely attached, via a fixation mechanism, to the second support layer.

### RELATED APPLICATION

This application claims the benefit of U.S. Provisional Patent Application Ser. No. 62/088,754, filed Dec. 8, 2014, the entirety of which is hereby incorporated by references for all purposes.

### TECHNICAL FIELD

The present disclosure relates generally to soft tissue implants, and more particularly to tissue repair implants that can be used to improve injured or otherwise defective target tissue within a subject.

### BACKGROUND

Biologic extracellular matrix products have shown utility in challenging hernia repair cases where contaminated or infected tissue is present. The durability of the repair is low, however, with a very high recurrence rate. Synthetic surgical mesh products, on the other hand, do not perform well in contaminated or infected tissue, but synthetic products have a much lower recurrence rate when placed in clean (non-contaminated) tissue. In addition, synthetic open pore surgical meshes can promote adhesion of the organs to the mesh as the hernia heals, which is undesirable.

### SUMMARY

The present disclosure relates generally to soft tissue implants, and more particularly to tissue repair implants that can be used to improve injured or otherwise defective target tissue within a subject.

One aspect of the present disclosure relates to a tissue repair implant comprising a first layer of extracellular matrix (ECM) and a second support layer of biocompatible material securely attached to the first layer at one or more fixation points. At least one of the fixation points comprises a first projection that is associated with the first layer and securely attached, via a fixation mechanism, to the second support layer.

Another aspect of the present disclosure relates to a method for forming a tissue repair implant. One step of the method can include forming a first projection in a first layer of ECM. Next, a second support layer of biocompatible material can be provided. The first projection can then be mated with the second support layer via a fixation mechanism.

Another aspect of the present disclosure relates to a method for repairing a target tissue in a subject. One step of the method can include providing a tissue repair implant comprising a first layer of ECM and a second support layer of biocompatible material securely attached to the first layer at one or more fixation points. At least one of the fixation points can include a first projection that is associated with the first layer and securely attached to the second support layer via a fixation mechanism. The tissue repair implant can be optionally shaped based on one or more anatomical and/or physiological characteristics of the target tissue. The tissue repair implant can then be implanted in the subject so that at least a portion of the second support layer contacts the target tissue.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1A is a perspective view showing a tissue repair implant, in an exploded configuration, constructed in accordance with one aspect of the present disclosure;

FIG. 1B is a cross-sectional view taken along Line 1B-1B in FIG. 1A;

FIG. 2A is a perspective view showing the tissue repair implant in FIGS. 1A-B in an assembled configuration;

FIG. 2B is a cross-sectional view taken along Line 2B-2B in FIG. 2A;

FIG. 2C is a plan view showing an entire tissue repair implant as depicted in FIGS. 1A-2B;

FIG. 2D is a perspective view showing a fixation point of the tissue repair implant in FIG. 2C;

FIG. 2E is a cross-sectional view taken along Line 2E,F-2E,F in FIG. 2D and showing initial suture attachment;

FIG. 2F is a cross-sectional view taken along Line 2E,F-2E,F in FIG. 2D and showing the suture (in FIG. 2E) pulled tight (in a final state);

FIG. 2G is an image showing the tissue repair implant in FIGS. 2A-F;

FIG. 2H is an image showing the mesothelium surface of the tissue repair implant in FIG. 2G;

FIG. 2I is a magnified image showing a fixation point of the tissue repair implant in FIGS. 2G-H;

FIG. 2J is a magnified image of a projection comprising the fixation point in FIG. 2I;

FIG. 3A is a perspective view showing a tissue repair implant, in an exploded configuration, constructed in accordance with another aspect of the present disclosure;

FIG. 3B is a cross-sectional view taken along Line 3B-3B in FIG. 3A;

FIG. 4A is a perspective view showing the tissue repair implant in FIGS. 3A-B in an assembled configuration;

FIG. 4B is a cross-sectional view taken along Lines 4B-4B in FIG. 4A;

FIG. 5A is a perspective view showing a tissue repair implant, in an exploded configuration, constructed in accordance with another aspect of the present disclosure;

FIG. 5B is a cross-sectional view taken along Line 5B-5B in FIG. 5A;

FIG. 6A is a perspective view showing the tissue repair implant in FIGS. 5A-B in an assembled configuration;

FIG. 6B is a cross-sectional view taken along Lines 6B-6B in FIG. 6A;

FIG. 7A is a magnified image showing a fixation point of the tissue repair implant in FIGS. 5A-6B;

FIG. 7B is an image showing a mesothelium surface of the tissue repair implant in FIGS. 5A-6B;

FIG. 8A is an image showing the tissue repair implant of FIGS. 5A-6B with a continuous suture extending through each of the fixation points;

FIG. 8B is a magnified image of the tissue repair implant in FIG. 8A;

FIG. 9A is an image showing the tissue repair implant in FIGS. 5A-6B with a continuous suture extending through a portion of the fixation points, as well as a series of interrupted sutures extending through the remaining portion of fixation points;

FIG. 9B is an image showing the mesothelium surface of the tissue repair implant in FIG. 9A;

FIG. 10 is a process flow diagram illustrating a method for forming a tissue repair implant according to another aspect of the present disclosure;

FIG. 11 is an image showing first and second plates used to form a first layer of extracellular matrix (ECM) according to the method of FIG. 10;

FIG. 12 is a magnified image of a first layer formed by the method of FIG. 10;

FIG. 13 is a magnified image showing the mesothelium surface of the first layer in FIG. 12; and

FIG. 14 is a process flow diagram illustrating a method for repairing a target tissue in a subject according to another aspect of the present disclosure.

### DETAILED DESCRIPTION

Definitions

Unless defined otherwise, all technical and scientific terms used herein have the same meaning as is commonly understood by one of skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the apparatus in use or operation in addition to the orientation depicted in the figures. For example, if the apparatus in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

As used herein, the term “biocompatible”, when used to describe a material, can refer to a material that is associated with (e.g., resides next to) biological tissue without harming the biological tissue to any appreciable extent.

As used herein, the terms “extracellular matrix” or “ECM” can refer to naturally-derived collagenous ECMs isolated from suitable animal or human tissue sources. Suitable ECMs can include, for instance, submucosa (e.g., small intestinal submucosa, stomach submucosa, urinary bladder submucosa, or uterine submucosa, each of these isolated from juvenile or adult animals), renal capsule membrane, amnion, dura mater, pericardium, serosa, dermis, and peritoneum or basement membrane materials, including liver basement membrane or epithelial basement membrane materials. These materials may be isolated and used as intact natural sheet forms, or reconstituted collagen layers including collagen-derived from these materials and/or other collagenous materials may be used.

As used herein, the terms “subject” and “patient” can be used interchangeably and refer to any warm-blooded organism including, but not limited to, human beings, pigs, rats, mice, dogs, goats, sheep, horses, monkeys, apes, rabbits, cattle, etc.

As used herein, the term “acellular” can mean free or essentially free from living cells.

As used herein, the term “substantially devoid of cells and cell components” can mean free or essentially free from cells (living or dead) and of cell membranes and other cell remnants. An ECM substantially devoid of cells or cell components can include the ECM membrane carrying cells or cell components at a level sufficiently low to be non-immunogenic when the ECM is implanted in a subject, especially a subject to which the cells or cell components are xenogeneic or allogeneic.

Overview

The present disclosure relates generally to soft tissue implants, and more particularly to tissue repair implants that can be used to improve injured or otherwise defective target tissue within a subject. Unlike conventional synthetic and biologic ECM products, the present disclosure provides a tissue repair implant that combines the properties of these two components to advantageously achieve the strength and durability of the synthetic material while benefitting from reduced foreign body reaction and the protection against adhesion during the healing process provided by the ECM material. The tissue repair implant allows time for a patient's own cells to propagate across (and into) the ECM material while the material comprising the ECM undergoes a remodeling process. At the same time, the biocompatible material undergoes a traditional healing response marked by an acute inflammatory phase, fibroplasia, and remodeling. The tissue repair implant also includes a number of discrete fixation points that securely join the ECM and biocompatible materials. The tissue fixation points, in combination with the ECM material, advantageously minimize the risk of an adverse foreign body reaction to the biocompatible material. Thus, by the time the ECM material is remodeled, the patient's own tissue has sufficiently grown across the biocompatible material such that adhesion risks are minimized by the development of a mesothelial tissue layer.

Tissue Repair Implants

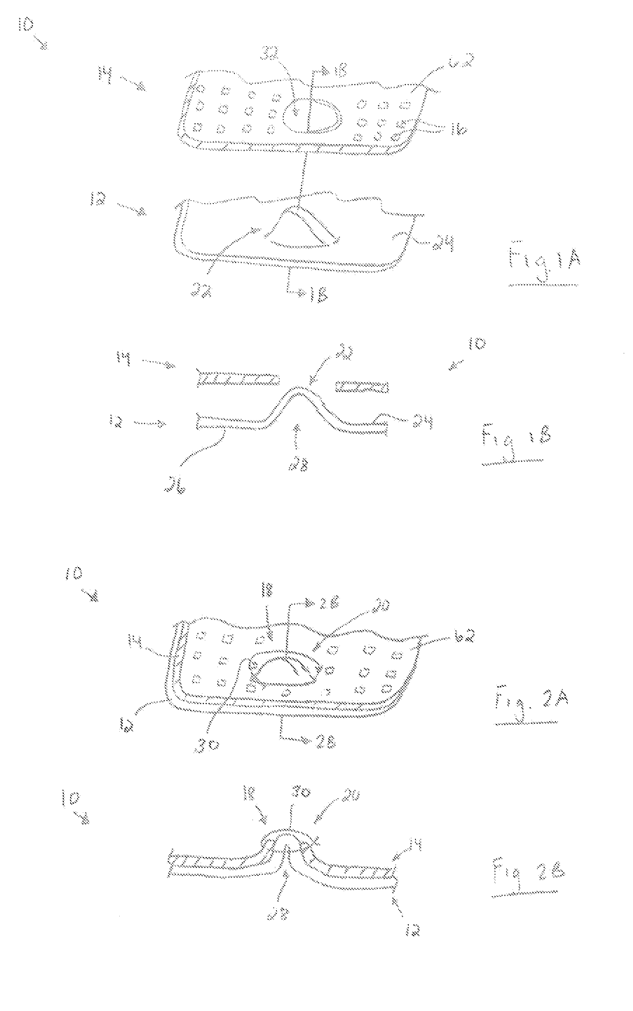

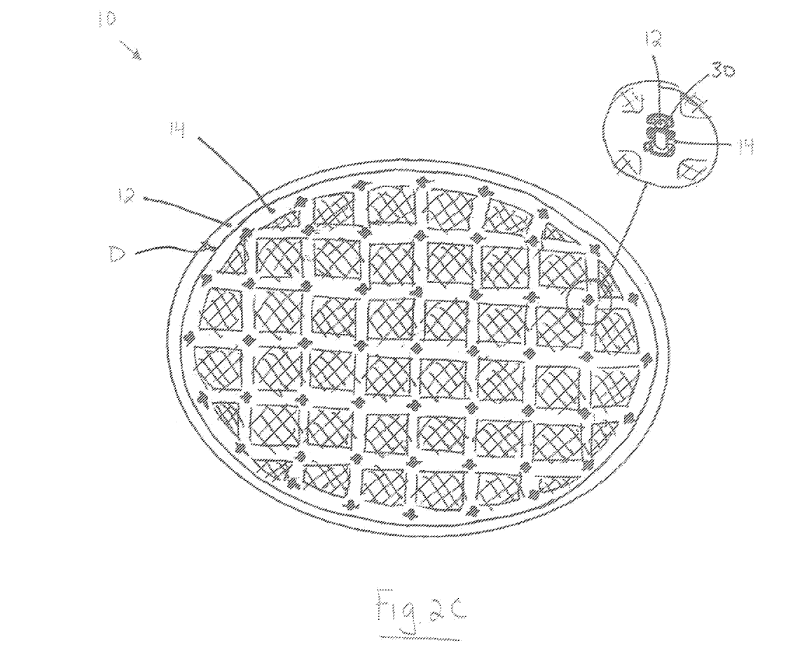

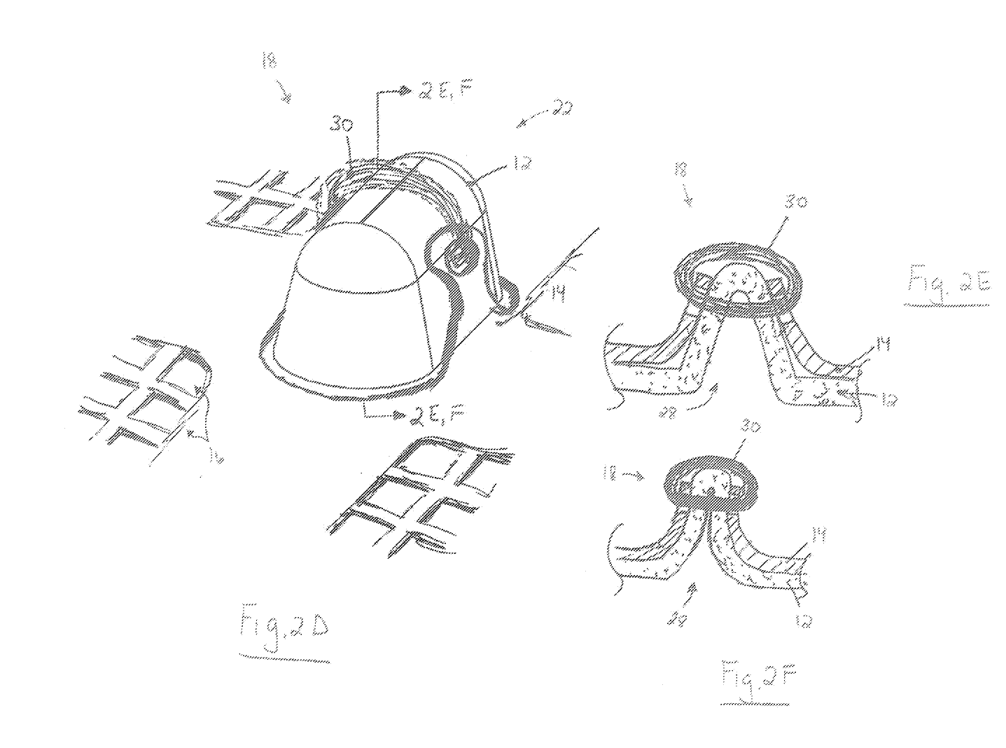

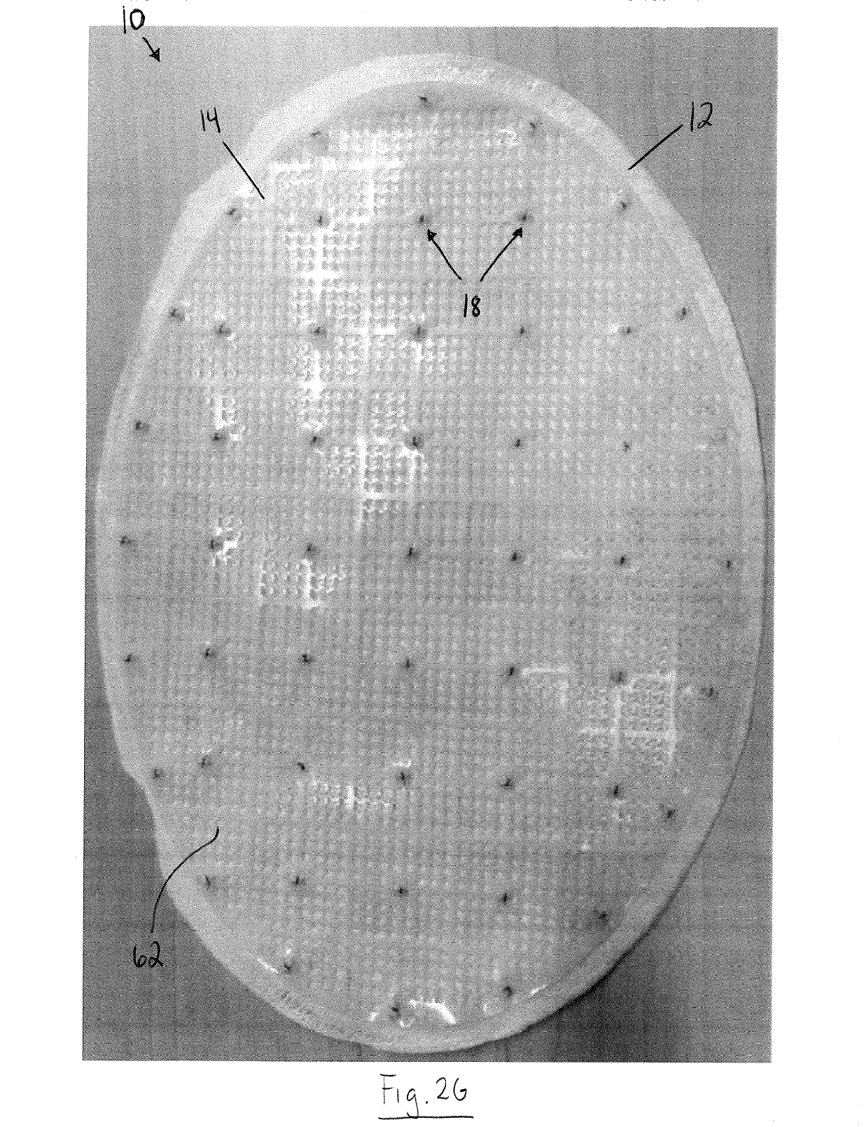

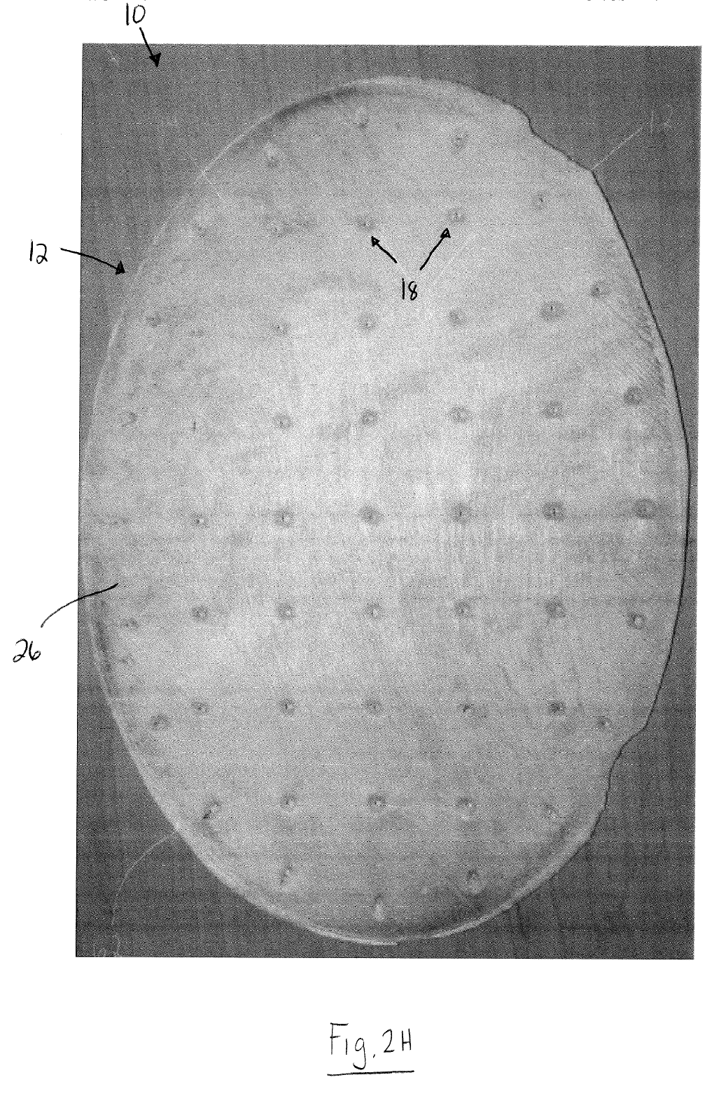

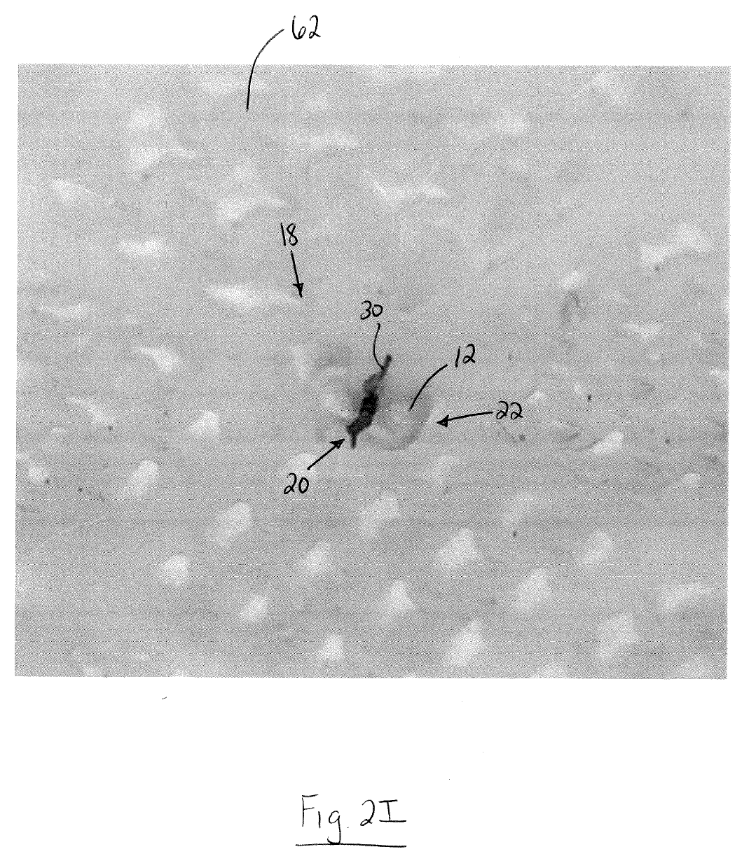

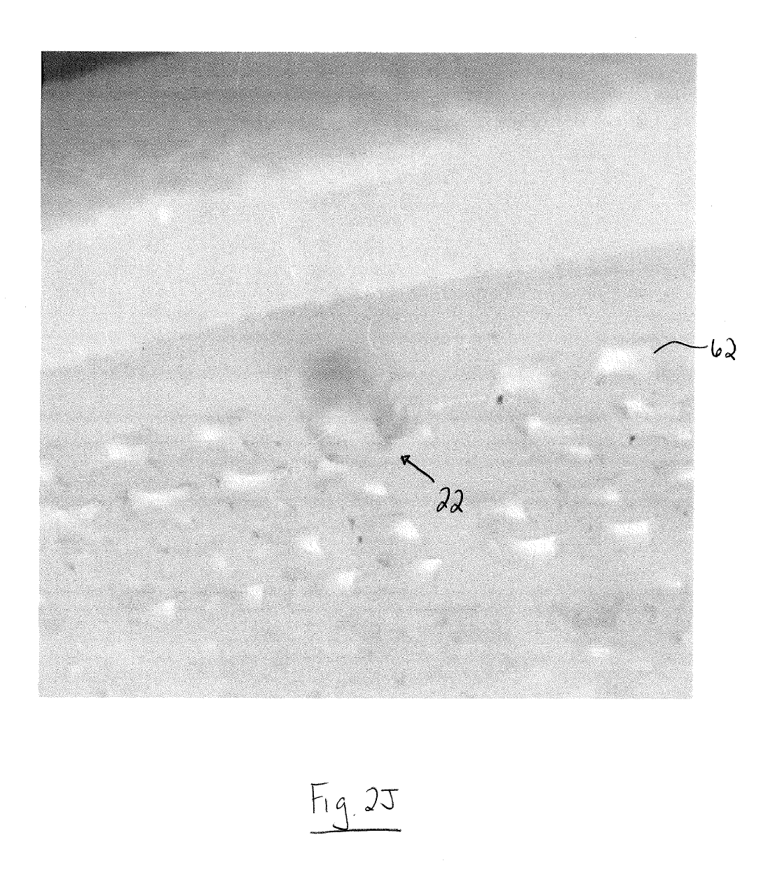

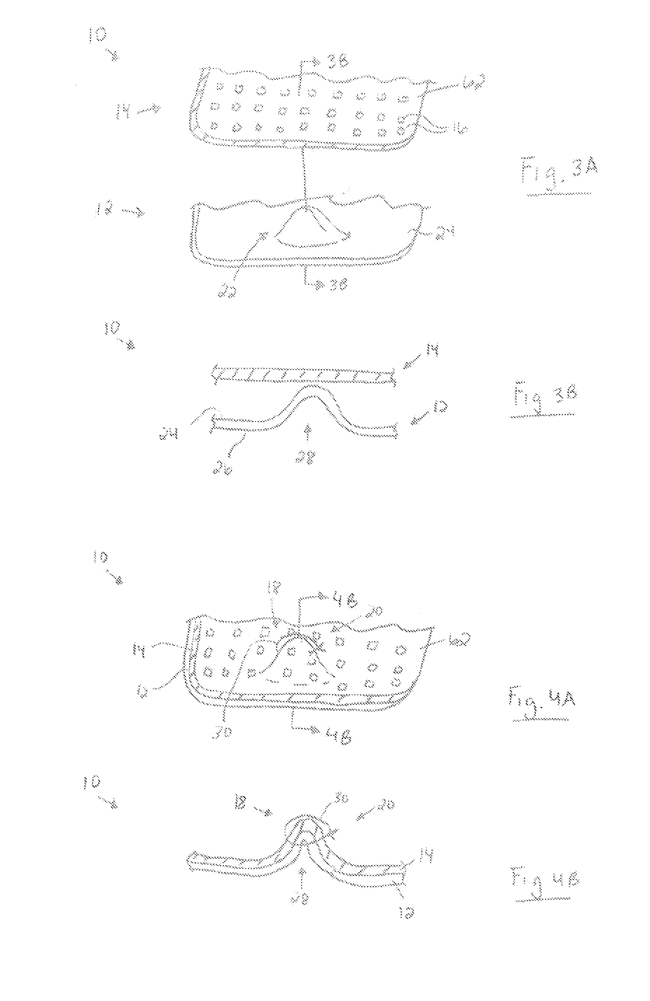

One aspect of the present disclosure can include a tissue repair implant 10 (FIGS. 1A-2B). The tissue repair implant 10 can have a multi-layer configuration (e.g., a bi-layer configuration) and be preformed for implantation into a subject. As shown in FIGS. 1A-B, the tissue repair implant 10 can comprise a first layer 12 of ECM and a second support layer 14 of biocompatible material that is securely attached to the first layer. In some instances, the first layer 12 can be acellular or substantially devoid of cells and cell components. The first layer 12 can be xenogeneic, allogeneic, or autologous. In some instances, the first layer 12 can be an ECM membrane. For example, the ECM membrane can be a peritoneal membrane derived from a non-human source, such as a pig. The length, width, and thickness of the first layer 12 can be sized and dimensioned based on the particular clinical need(s) of a subject.

The second support layer 14 can be made of a biocompatible material (or combination of materials) that is non-resorbable or has a resorption time greater than a resorption time of the first layer 12. Advantageously, the second support layer 14 can be configured to impart the tissue repair implant 10 with a desired degree of tensile strength and/or rigidity, in turn providing a durable tissue repair implant. The second support layer 14 can be made of any one or combination of synthetic and/or naturally-derived materials. In some instances, the second support layer 14 can be made of a cross-linked ECM. In other instances, the second support layer 14 can additionally or alternatively be made of one or a combination of non-resorbable polymers, such as polypropylene, polyethylene, polyethylene terephthalate, polytetrafluoroethylene (PTFE), polyaryletherketone, nylon, fluorinated ethylene propylene, polybutester, and silicone, or copolymers thereof (e.g., a copolymer of polypropylene and polyethylene). In one example, the second support layer 14 can be made of condensed PTFE. In further instances, the second support layer 14 can additionally or alternatively be made of one or a combination of resorbable polymers, such as polyglycolic acid (PGA), polylactic acid (PLA), polycaprolactone, and polyhydroxyalkanoate, or copolymers thereof (e.g., a copolymer of PGA and PLA). The polymers can be of the D-isoform, the L-isoform, or a mixture of both.

The second support layer 14 can have a porous configuration to support tissue in-growth. For example, the second support layer 14 can include a plurality of cell openings, each of which defines a pore 16. Each pore 16 can vary in size and/or shape and be uniform or non-uniform within the second support layer 14. Specific examples of variations in pore size and/or shape and/or spatial arrangement are disclosed in U.S. Pat. No. 8,796,015 to Gingras.

In some instances, the second support layer 14 can include one or more therapeutic agents (e.g., growth factors) associated therewith (e.g., disposed on or imbibed within the second support layer). Suitable growth factors can include cytokines, interleukins, and other peptide growth factors, such as bone morphogenetic proteins (BMPs), epidermal growth factor (EGF), members of the fibroblast growth factor (FGF) family, platelet-derived growth factor (PDGF), nerve growth factor (NGF), glial growth factor (GGF), vascular endothelial growth factor (VEGF), or members of the transforming growth factor (TGF) family (e.g., TGF-α or TGF-β). Alternatively or in addition, the second support layer 14 can include one or more types of biological cells (e.g., stem cells, progenitor cells, e.g., osteoblasts or any other partially differentiated cell), cells of an established cell line, or mature cells such as fibroblasts) associated therewith (e.g., disposed on or imbibed within the second support layer). Other agents that may be additionally or optionally associated with the second support layer 14 can include antibiotics, antiviral agents, antifungal agents, and/or vitamins or minerals.

The second support layer 14 can be securely attached to the first layer 12 at one or more fixation points 18 via a fixation mechanism 20. Each of the fixation points 18 can comprise a first projection 22 that is associated with the first layer 12 (e.g., formed from the first layer) and securely attached, via the fixation mechanism 20, to the second support layer 14. The first projection 22 can include a preformed, three-dimensional structure that protrudes from a first major surface 24 of the first layer 12. Although a projection 22 having a dome-shaped configuration is depicted in FIGS. 1A-B, it will be appreciated that any other shape or configuration is possible. The first projection 22 can be defined by a portion of the first major surface 24 and an oppositely disposed portion of a second major surface 26 (also referred to as the mesothelium surface). The second major surface 26 that forms a part of the first projection 22 can resemble a dimple-like structure that defines a cavity 28. Advantageously, when the first layer 12 and the second support layer 14 are securely joined by the fixation mechanism 20 (e.g., a suture 30), the cavity 28 is partly or entirely collapsed, thereby minimizing or eliminating the degree to which the fixation mechanism is exposed within the body of the subject (e.g., to minimize contact with an internal organ).

In some instances, the fixation mechanism 20 can include a suture 30 arranged in an interrupted or continuous manner (e.g., extending across several fixation points 18). It will be appreciated, however, that the fixation mechanism 20 can also include a friction fit, adhesive, or other type of medical fastener sufficient to securely attach the first layer 12 to the second support layer 14. For example, in some instances, the fixation mechanism 20 can include clips, pins, or the like. The fixation mechanism 20 can be made of a biocompatible, non-resorbable material (e.g., PGA). Alternatively, the fixation mechanism 20 can be made from a biocompatible and/or resorbable material (or materials), such as those disclosed by Pillai and Sharma, J Biomat Applications 25:291-366 (November 2010).

In one example, the fixation mechanism 20 can be sized and dimensioned in an identical or similar manner as an automotive interior panel retainer (not shown). Exemplary retainers are disclosed in U.S. Pat. No. 6,196,607, and are commercially available from Clipsandfasteners.com, Inc. (Anaheim, Calif.), for instance. In use, such a retainer can be pressed through a projection 22 in a transverse manner to securely join the first and second layers 12 and 14. Additionally or optionally, a retainer can include a U-shaped clip having a first end that is directly attached to the retainer and a second female end that is initially free from contact with the retainer. When the retainer is pressed through a projection 22, a male end of the retainer can securely mate with the second female end of the retainer to secure the retainer about the projection.

In another example, the fixation mechanism 20 can be sized and dimensioned as a hog ring (not shown). Examples of hog rings are known in the art and are commercially available from VER Sales, Inc. (Burbank, Calif.), for instance. In use, an appropriately-sized hog ring can be squeezed through a projection 22 in a transverse manner and then cinched together to securely join the first and second layers 12 and 14.

In another example, the fixation mechanism 20 can be sized and dimensioned as a star lock fastener (not shown). Examples of star lock fasteners are known in the art and commercially available from Titgemeyer Ltd. (Tipton, GB), for instance. In use, an appropriately-sized star lock fastener can be used to securely join the first and second layers 12 and 14 by pushing a projection 22 up into the “star”, whereafter the “star” retains the first and second layers 12 and 14 comprising the projection from the top side with no part of the “star” being exposed on the second major surface 26 of the first layer 12 (e.g., so that the “star” is free from direct contact with an internal organ of a subject).

One example of a tissue repair implant 10 is illustrated in FIGS. 1A-2F. In this example, the second support layer 14 can include a plurality of pores 16 between which is/are interspersed one or more preformed, complementary pores 32. Each of the complementary pores 32 can be sized and dimensioned to receive all or a portion of a projection 22. As shown in FIG. 1A, for example, the complementary pore 32 can have a circular cross-section that is sized and dimensioned to receive the first projection 22. When the second support layer 14 is applied to the first layer 12 (FIG. 2A), the second support layer can be nested around each of the projections 22 such that at least a portion of the second support layer overlaps with a portion of the first projection. The overlapping portion permits the second support layer 14 to be securely attached to the first layer 12 (e.g., the first projection 22) via the fixation mechanism 20. Then, when the fixation mechanism 20 (e.g., a suture 30) is applied to the tissue repair implant 10 (FIG. 2B), the cavity 28 is collapsed to minimize or eliminate exposure of the suture 30 on the second major surface 26 of the first layer 12 (e.g., so that the suture is free from direct contact with an internal organ of a subject). As shown in FIG. 2C, the first layer 12 can extend a distance D (e.g., about 5 mm) beyond the edge of the second support layer 14. The material spanning the distance D can assist in attaching the tissue repair implant 10 at a surgical repair site by providing adhesion protection from the edge of the second support layer 14.

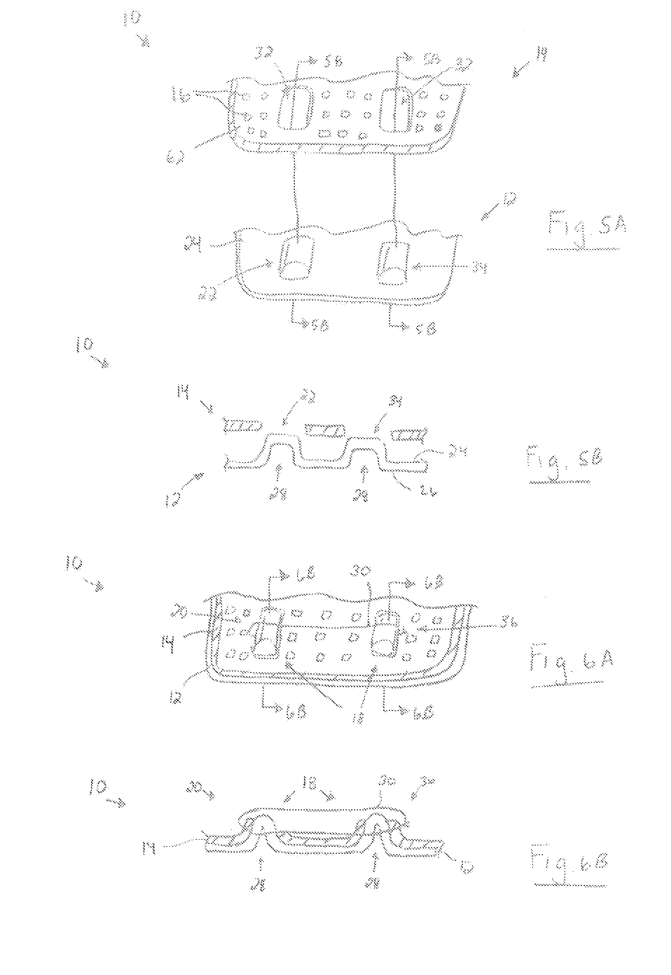

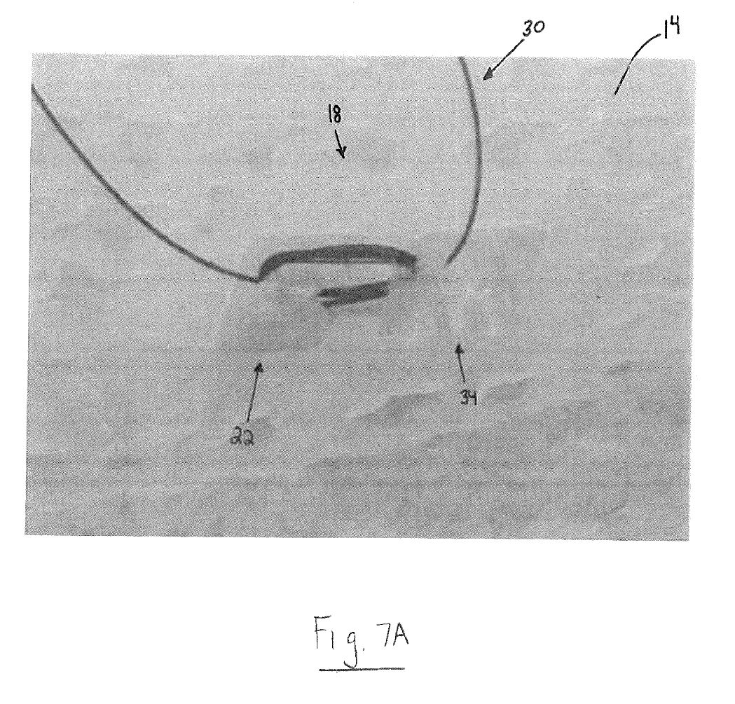

Another example of a tissue repair implant 10 is illustrated in FIGS. 3A-4B. In this example, the second support layer 14 can comprise a single, continuous sheet without any preformed complementary pores 32 (FIGS. 3A-B). The second support layer 14 can be laid over the first layer 12 so that each projection 22 is covered thereby. Once the second support layer 14 has been placed over the first layer 12, the first layer and the second support layer can be securely attached to one another via a suitable fixation mechanism 20 (FIGS. 4A-B).

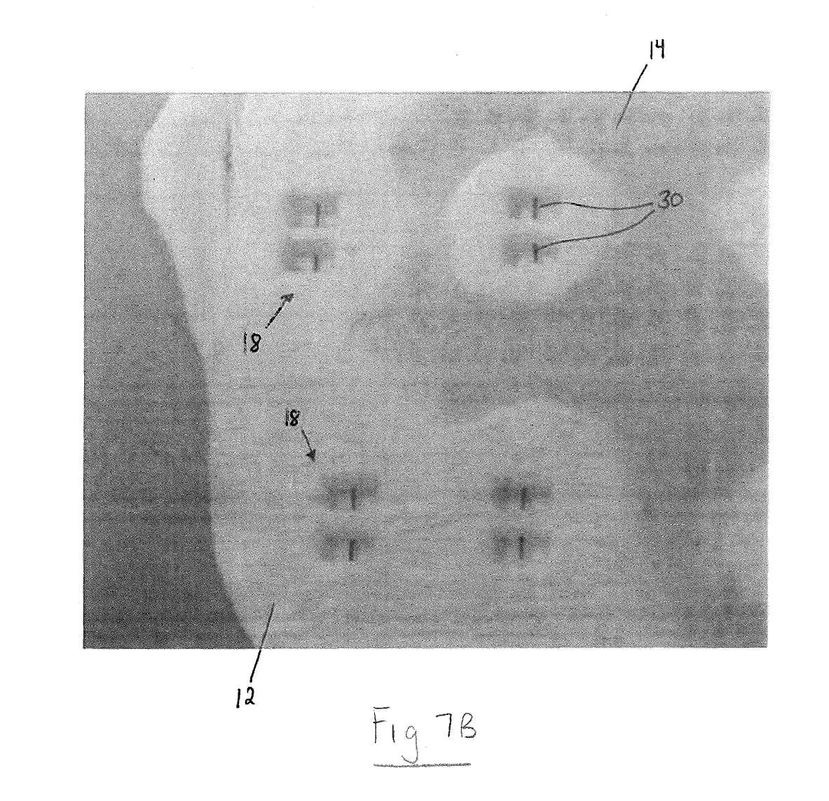

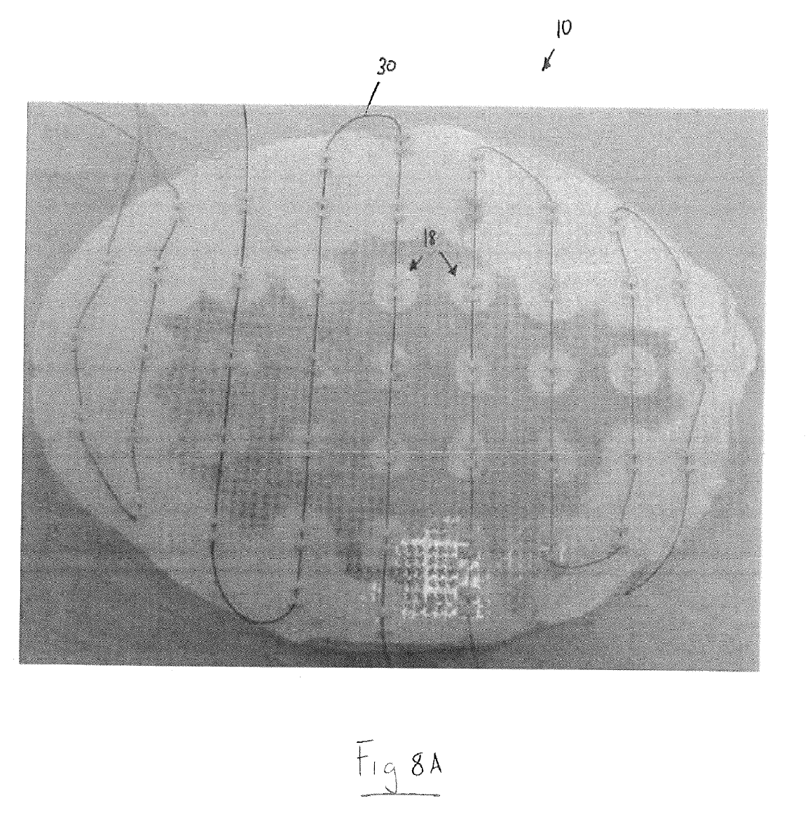

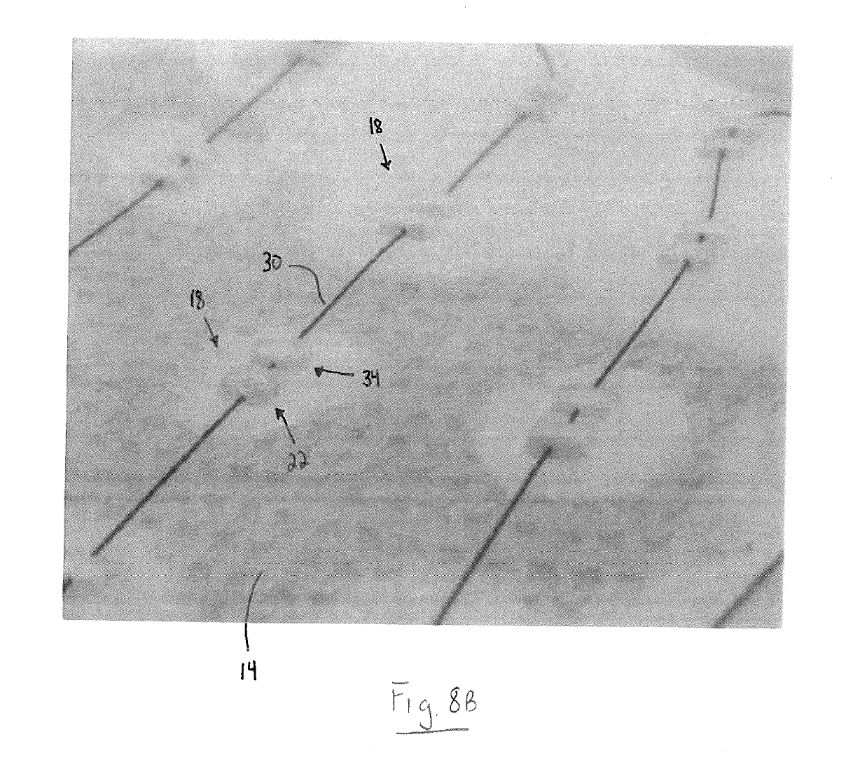

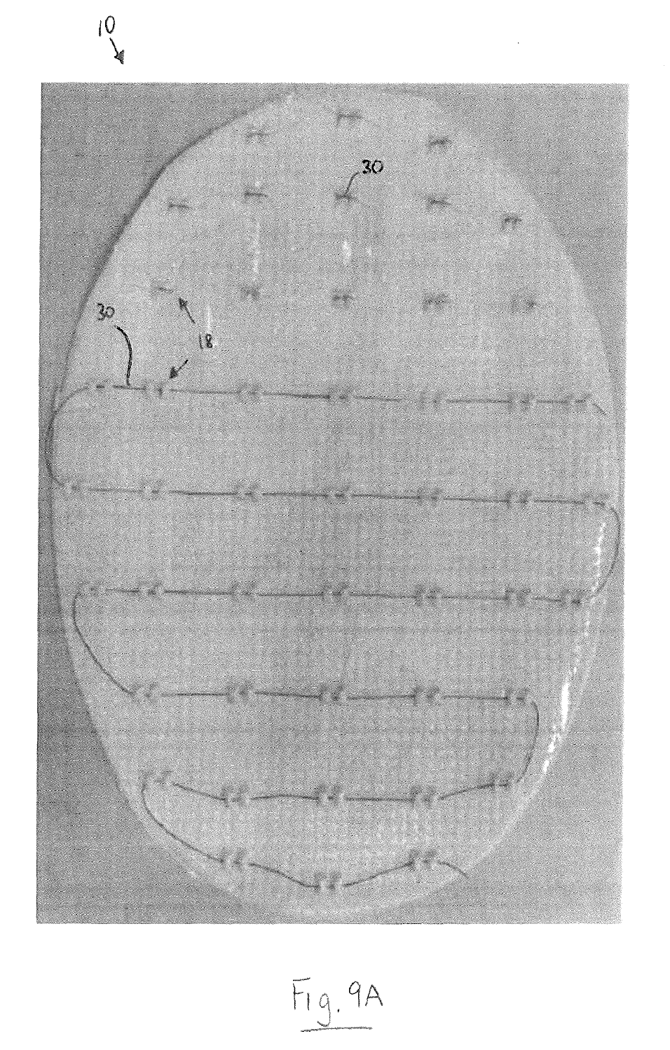

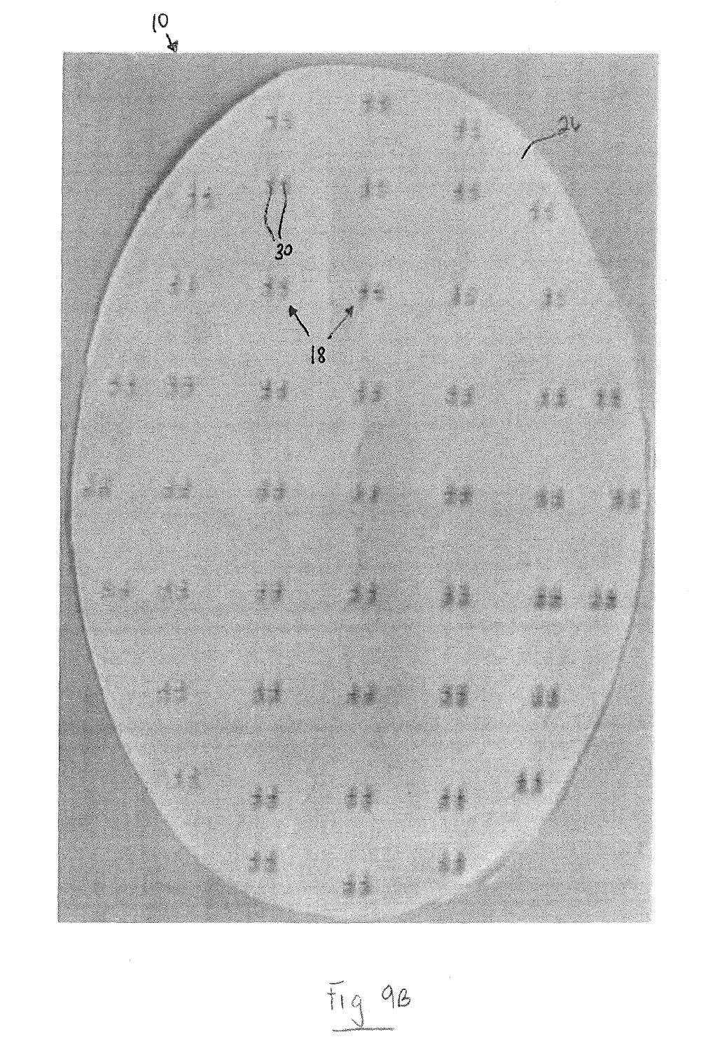

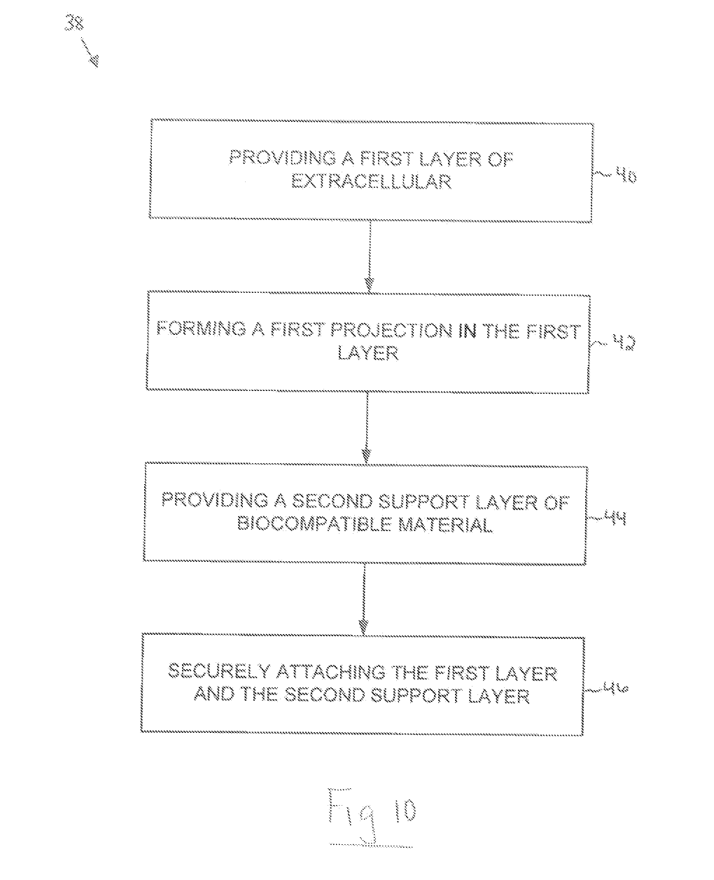

Another example of a tissue repair implant 10 is illustrated in FIGS. 5A-6B. In this example, each fixation point 18 can comprise a preformed first projection 22 that is located immediately adjacent, and substantially parallel to, a second projection 34. Each of the first and second projections 22 and 34 can have a rectangular three-dimensional structure. Like the first projection 22, the second projection 34 is associated with the first layer 12. The second support layer 14 can comprise oppositely disposed complementary pores 16 adapted to receive the first and second projections 22 and 34. The second projection 34 can be attached to the second support layer 14 via a second fixation mechanism 36. The second fixation mechanism 36 can be the same (e.g., shared) or different (e.g., physically separate) than the fixation mechanism 20 used to attach the first projection 22 to the second support layer 14. As shown in FIGS. 6A-B, for example, the first and second fixation mechanisms 20 and 36 can be shared and comprise a continuous suture 30 that securely joins only the second projection 34 and the second support layer 14.

Formation

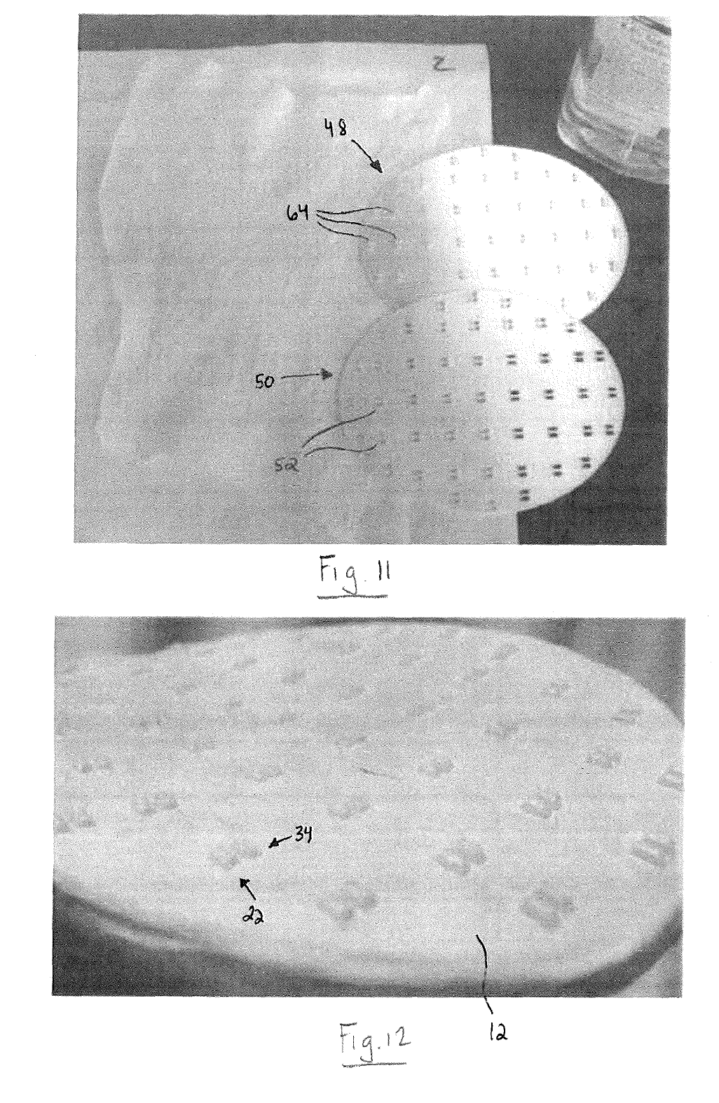

Another aspect of the present disclosure can include a method 38 (FIG. 10) for forming a tissue repair implant 10. As shown in FIG. 10, the method 38 can include the steps of: providing a first layer 12 of ECM (Step 40); forming a first projection 22 in the first layer (Step 42); providing a second support layer 14 of biocompatible material (Step 44); and securely attaching the first layer and the second support layer to form the tissue repair implant (Step 46).

At Step 40, an appropriate amount of tissue comprising an ECM can be harvested from a subject and used to form the first layer 12. For example, a desired amount of peritoneum from a pig can be harvested. The harvested tissue can then be processed to obtain an acellular ECM or an ECM that is substantially devoid of cells and cell components. In one example, Step 40 of the method 38 can include obtaining a first layer 12 of ECM membrane comprising decellularized porcine peritoneal membrane.

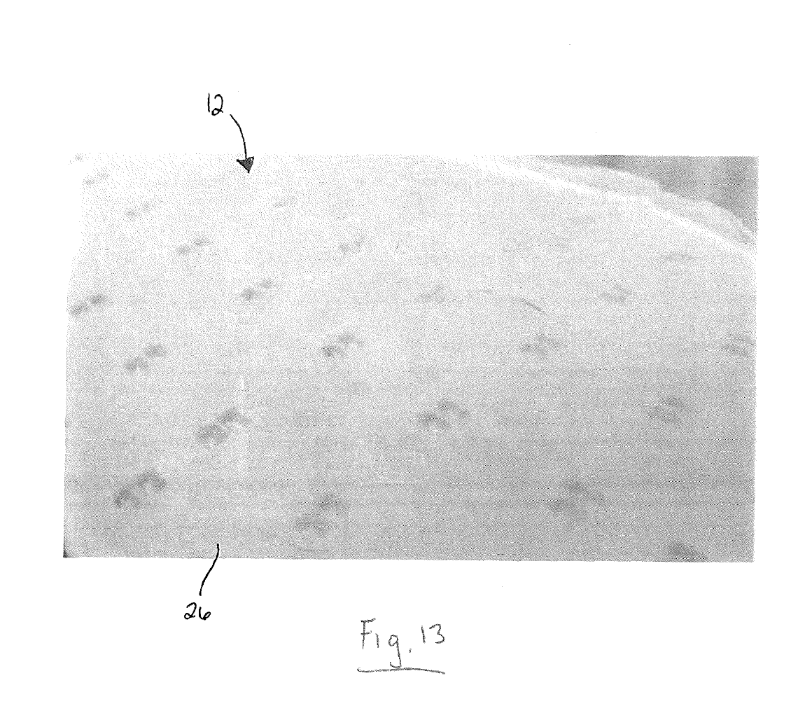

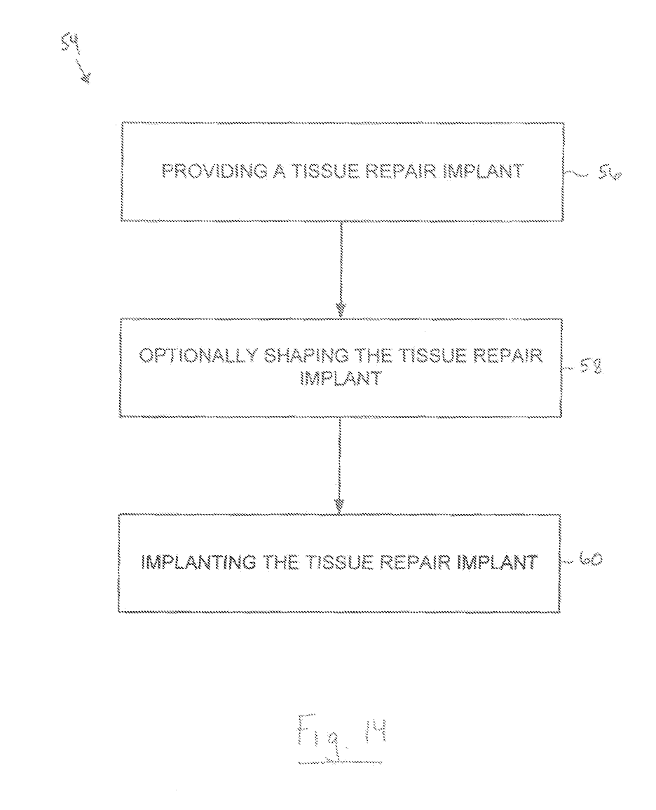

A first projection 22 can then be formed in the first layer 12 (Step 42). To do so, the first layer 12 can be placed between first and second plates 48 and 50 (FIG. 11). The first plate 48 can include a number of teeth 64 that extend beyond a major surface thereof. The second plate 50 can include a number of apertures 52, which are configured to receive the teeth 64 when the first plate 48 is mated with the second plate. The size, shape, and spatial arrangement of each tooth can be predetermined so as to form a corresponding projection 22 with a complementary size, shape, and spatial arrangement. At Step 42, the first layer 12 is placed between the first and second plates 48 and 50, whereafter pressure can be applied to one or both of the plates in an amount and for a time sufficient to form one or more projections 22 (FIGS. 12-13).

At Step 44, a second support layer 14 of biocompatible material can be provided. The second support layer 14 can be formed by one or a combination of techniques, such as laser machining, die punching, water jet cutting, chemical etching, textile processing/knitting, and the like. Depending upon the configuration of the second support layer 14 (such as those described above), the second support layer can then be securely attached to the first layer 12 via a fixation mechanism 20. Following Step 46, the tissue repair implant 10 can be shaped and dimensioned for a particular application. Alternatively, the first layer 12 and/or the second support layer 14 can be shaped and dimensioned for a particular application prior to assembly into the tissue repair implant 10.

It will be appreciated that the method 38 can involve techniques other than the one discussed above for forming the first layer 12. For example, the projections 22 can be formed by skiving.

Methods of Use

Another aspect of the present disclosure can include a method 54 (FIG. 14) for repairing a target tissue in a subject. The method 54 can include the steps of: providing a tissue repair implant 10 (Step 56); optionally shaping the tissue repair implant (Step 58); and implanting the tissue repair implant (Step 60). The method 54 can find use for a variety of clinical indications, including ventral hernia repair and reconstruction of soft tissue.

Step 56 of the method 54 can include providing a tissue repair implant 10. The tissue repair implant 10 can be prepared in an identical or similar manner as described above. At Step 58, the tissue repair implant 10 can be optionally shaped prior implantation to accommodate the particular target tissue.

Once the tissue repair implant 10 has been appropriately prepared, the tissue repair implant can be implanted in the subject (Step 60). The particular implantation technique (e.g., endoscopic, intra-peritoneal onlay mesh repair, etc.) will depend upon the nature and location of the target tissue. Once the tissue repair implant 10 has been appropriately positioned in the subject, the tissue repair implant can be sutured or stapled in place (e.g., to close or reinforce a defect) so that the first layer 12 is oriented with its mesothelium surface 26 oriented in an outward direction, and a first major surface 62 of the second support layer 14 is oriented towards the target tissue to facilitate tissue in-growth and create a durable repair. Securing the tissue repair implant 10 in this manner advantageously allows the mesothelium surface 26 to act as an adhesion barrier while also mitigating the host foreign body response.

From the above description of the present disclosure, those skilled in the art will perceive improvements, changes and modifications. Such improvements, changes, and modifications are within the skill of those in the art and are intended to be covered by the appended claims. All patents, patent applications, and publication cited herein are incorporated by reference in their entirety.

The following is claimed:

1. A tissue repair implant comprising:
a first layer of extracellular matrix (ECM); and
a second support layer of biocompatible material securely attached to the first layer at one or more fixation points, the second support layer being non-resorbable or having a resorption time greater than a resorption time of the ECM;
wherein at least one of the fixation points comprises a first projection that is associated with the first layer and securely attached, via a fixation mechanism, to the second support layer.

2. The tissue repair implant of claim 1, wherein the ECM is xenogeneic.

3. The tissue repair implant of claim 1, wherein the ECM is an ECM membrane.

4. The tissue repair implant of claim 3, wherein the ECM membrane is a peritoneal membrane.

5. The tissue repair implant of claim 1, wherein the first layer is oriented with its mesothelium surface oriented in an outward direction to generate one or more organ contacting surfaces.

6. The tissue repair implant of claim 1, wherein the second support layer is configured to promote in-growth of tissue.

7. The tissue repair implant of claim 1, wherein the second support layer is a synthetic material.

8. The tissue repair implant of claim 1, wherein the second support layer is a cross-linked ECM.

9. The tissue repair implant of claim 1, wherein the fixation mechanism comprises one or more interrupted sutures extending through each of the first layer and the second support layer.

10. The tissue repair implant of claim 1, wherein the fixation mechanism comprises a continuous suture extending through each of the first layer and the second support layer.

11. The tissue repair implant of claim 1, wherein the fixation mechanism includes a clip, a pin, an adhesive, or a combination thereof.

12. The tissue repair implant of claim 1, wherein the second support layer includes a pre-formed, complementary pore configured to receive the first projection.

13. The tissue repair implant of claim 11, wherein the one or more interrupted sutures is/are free from direct contact with an organ when the tissue repair implant is implanted in a subject.

14. The tissue repair implant of claim 1, wherein one or more of the fixation points further comprises a second projection that is associated with the first layer and securely attached to the second support layer via a shared second fixation mechanism;
wherein the second projection is located immediately adjacent, and substantially parallel to, the first projection.

15. A method for forming a tissue repair implant, the method comprising the steps of:
forming a first projection in a first layer of ECM;
providing a second support layer of biocompatible material; and
mating the first projection with the second support layer via a fixation mechanism.

16. The method of claim 15, wherein the first projection is mated with the second support layer using one or more sutures that extend through each of the first layer and the second support layer.

17. The method of claim 15, wherein the first projection is mated with the second support layer using one or a combination of clips, pins, and adhesives that join the first layer with the second support layer.

18. A method for repairing a target tissue in a subject, the method comprising the steps of:
providing a tissue repair implant comprising a first layer of ECM and a second support layer of biocompatible material securely attached to the first layer at one or more fixation points, at least one of the fixation points including a first projection that is associated with the first layer and securely attached to the second support layer via a fixation mechanism;
optionally shaping the tissue repair implant based on one or more anatomical and/or physiological characteristics of the target tissue; and
implanting the tissue repair implant in the subject so that at least a portion of the second support layer contacts the target tissue.

19. The method of claim 18, wherein the first projection is attached to the second support layer via one or more sutures that extend through each of the first layer and the second support layer.

20. The method of claim 18, wherein the first projection is attached to the second support layer via one or a combination of clips, pins, and adhesives that join the first layer with the second support layer.
